(19) United States
(12) Patent Application Publication
(10) Pub. No.: US 2026/0335616 A1
(43) Pub. Date: Oct. 29, 2026

(54) THERAPEUTIC SUBSTANCE MONITORING

(71) Applicant: Cochlear Limited, Macquarie University, NSW (AU)

(72) Inventors: Daniel SMYTH, Dublin (IE); Wolfram Frederik DUECK, Berlin (DE)

(21) Appl. No.: 19/111,495

(22) PCT Filed: Sep. 1, 2023
(86) PCT No.: PCT/IB2023/058684

§ 371 Date: Mar. 13, 2025

Related U.S. Application Data
(60) Provisional application No. 63/408,182, filed on Sep. 20, 2022.

(51) Int. Cl. A61M 31/00; A61N 1/36

(52) U.S. Cl. CPC A61M 31/002; A61M 2205/04; A61M 2205/054; A61M 2205/3317; A61M 2205/3368; A61M 2205/50; A61N 1/36038; A61N 1/36046

(57) ABSTRACT

Presented herein are techniques for monitoring a state, amount, and/or concentration of a therapeutic substance in, for example, an implantable component of an implantable medical device. In particular, an implantable component can include at least one therapeutic substance portion having a therapeutic substance therein for delivery to a recipient. The techniques presented herein measure the impedance across the at least one therapeutic substance portion and use the measured impedance to estimate the state, amount and/or concentration of the therapeutic substance within the at least one therapeutic substance portion.

### BACKGROUND

### Field of the Invention

The present invention relates generally to techniques for monitoring a state, amount and/or concentration of a therapeutic substance.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a system is provided. The system comprises: at least one therapeutic substance portion including a therapeutic substance; and a measurement system configured to measure an impedance across the at least one therapeutic substance portion and to estimate a state of the therapeutic substance within the at least one therapeutic substance portion based on the measured impedance across the at least one therapeutic substance portion.

In another aspect, a method is provided. The method comprises: measuring an impedance of at least one therapeutic substance portion including a therapeutic substance; and estimating, based on the measured impedance, a state of the therapeutic substance within the at least one therapeutic substance portion.

In another aspect, one or more non-transitory computer readable storage media are provided. The one or more non-transitory computer readable storage media comprise instructions that, when executed by a processor, cause the processor to: obtain an impedance of at least one therapeutic substance portion; and estimate, based on the impedance of the at least one therapeutic substance portion, an amount of at least one therapeutic substance remaining within the at least one therapeutic substance portion.

In another aspect, a system is provided. The system comprises: at least one therapeutic substance portion including a therapeutic substance configured to implantable in a body of a recipient; at least a first electrode and at least a second electrode configured to be implanted in the recipient proximate to the therapeutic substance; circuitry configured to measure an impedance between the at least first electrode and the at least second electrode; and at least one processor configured to estimate a concentration of the therapeutic substance in the therapeutic substance portion from the measured impedance.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a schematic diagram illustrating a therapeutic substance portion and measurement electrodes, in accordance with certain embodiments presented herein;

FIGS. 3A, 3B, 3C, and 3D are a series of schematic diagrams showing changes in impedance of the therapeutic substance portion of FIG. 2, in accordance with certain embodiments presented herein;

FIG. 4 is a schematic diagram illustrating another therapeutic substance portion and measurement electrodes, in accordance with certain embodiments presented herein;

FIG. 5 is a graph illustrating how changing impedance over time is indicative of a state, amount, and/or concentration of therapeutic substance in the therapeutic substance portion of FIG. 4;

FIG. 6 is a schematic diagram illustrating a therapeutic substance portion in the form of a reservoir, and measurement electrodes, in accordance with certain embodiments presented herein;

FIG. 7 is a schematic diagram illustrating a measurement system, in accordance with certain embodiments presented herein;

FIG. 8 is a flow chart illustrating an example method for monitoring a state, amount and/or concentration of a therapeutic substance in a therapeutic substance portion, in accordance with certain embodiments presented herein;

FIG. 9 is a flow chart illustrating another example method for monitoring a state, amount and/or concentration of a therapeutic substance in a therapeutic substance portion, in accordance with certain embodiments presented herein;

FIG. 10 is a flow chart illustrating another example method for monitoring a state, amount and/or concentration of a therapeutic substance in a therapeutic substance portion, in accordance with certain embodiments presented herein;

FIG. 11 is a flow chart illustrating yet another example method for monitoring a state, amount and/or concentration of a therapeutic substance in a therapeutic substance portion, in accordance with certain embodiments presented herein;

FIG. 12 is a schematic diagram illustrating a vestibular stimulator system with which aspects of the techniques presented herein can be implemented; and

FIG. 13 is a schematic diagram illustrating a retinal prosthesis system with which aspects of the techniques presented herein can be implemented.

### DETAILED DESCRIPTION

A growing area of research and development relates to the use of pharmaceutical compounds, biological substances, bioactive substances, etc., including pharmaceutical agents/active pharmaceutical ingredients (APIs), genes, messenger RNA (mRNA) or other signalling compounds that promote recovery and resolution, chemicals, ions, drugs, etc. to treat a variety of disorders within the body of individual patient/recipient. These various substances, which are collectively and generally referred to herein as “therapeutic substances,” are delivered to induce some therapeutic results/treatment within the body of the recipient. For example, therapeutic substances can be delivered to treat ear disorders (e.g., tinnitus, hearing loss, Ménière's disease, etc.), to treat infections post-surgery, to fight cancer cells, to treat neurodegenerative diseases, to treat infectious diseases, to regenerate neural tissue etc.

Presented herein are techniques for monitoring a state, amount, and/or concentration of a therapeutic substance in, for example, an implantable component of an implantable medical device. In particular, an implantable component can include at least one therapeutic substance portion having a therapeutic substance therein for delivery to a recipient. The techniques presented herein measure the impedance across the at least one therapeutic substance portion and use the measured impedance to estimate a state, amount, and/or concentration of the therapeutic substance within the at least one therapeutic substance portion.

Merely for ease of description, the techniques presented herein are primarily described with reference to a specific implantable medical device system, namely a cochlear implant system. However, it is to be appreciated that the techniques presented herein can also be partially or fully implemented by other types of implantable medical devices. For example, the techniques presented herein can be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

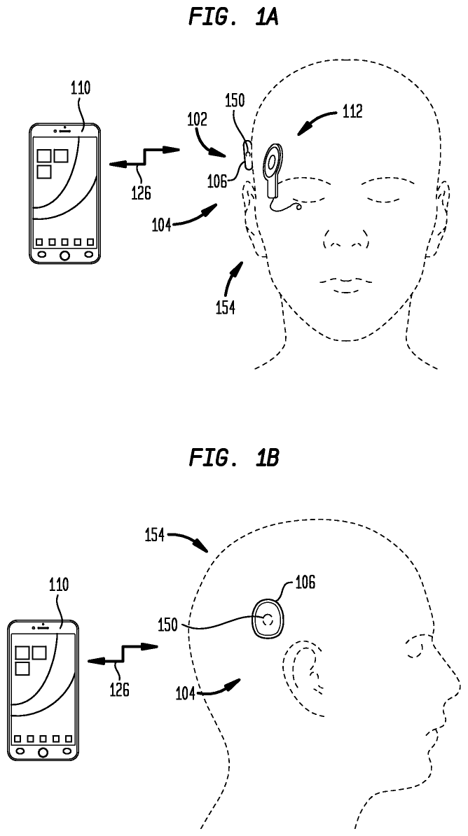

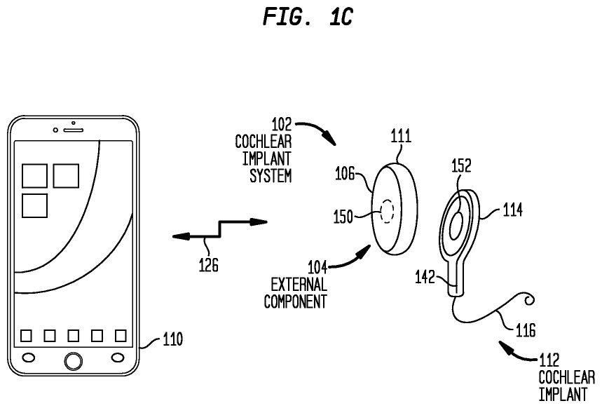

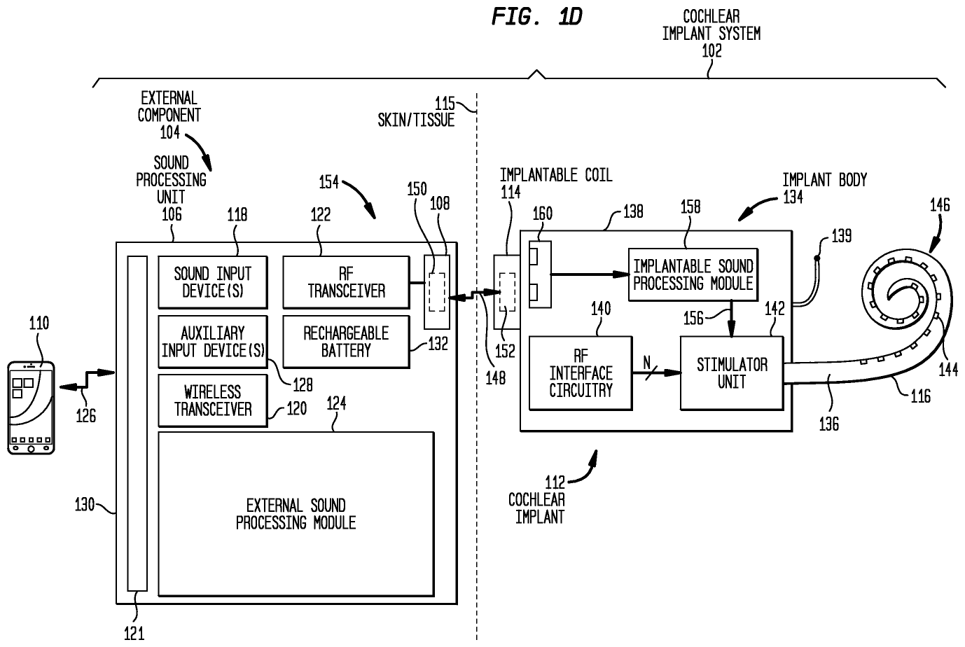

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component can comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear implant system 102 (e.g., OTE sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-ID, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices can include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a carrier member (e.g., a flexible silicone body) 115 with a plurality of longitudinally spaced intra-cochlear electrical stimulating contacts (electrodes) 144 disposed therein. The electrodes 144 collectively form a contact or electrode array 146 for delivery of electrical stimulation (current) to the recipient's cochlea. As shown, the stimulating assembly 116 also comprises one or more therapeutic substance delivery portions 162. The therapeutic substance delivery portion(s) 162 include/comprise one or more therapeutic substances for delivery to the recipient.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

As noted above, an implantable component, such as cochlear implant 112, can include at least one “therapeutic substance portion” or “drug containing portion (DCP)” that includes one or more therapeutic substances therein for delivery to a recipient. In the example of FIG. 1D, the stimulating assembly 116 includes two (2) therapeutic substance delivery portions 162. It is to be appreciated that this specific location and number of therapeutic substance delivery portions is merely illustrative and that, in the context of cochlear implant 112, therapeutic substance portions can be disposed in, for example, the implant body 134, the lead region 136, the stimulating assembly 116, etc. In addition, and as detailed below, therapeutic substance portions in accordance with embodiments presented herein can have a number of different arrangements. For example, in certain embodiments, the therapeutic substance portion can be a solid material (e.g., silicone) that is loaded/doped with a therapeutic substance. In other embodiments, the therapeutic substance portion can be a reservoir that includes a solid or liquid therapeutic substance.

As noted above, the techniques presented herein measure the impedance across a therapeutic substance portion and use the measured impedance to estimate a state, amount, and/or concentration of the therapeutic substance within the therapeutic substance portion. Example techniques for measuring impedance and estimating the therapeutic substance state, amount and/or concentration are described further below.

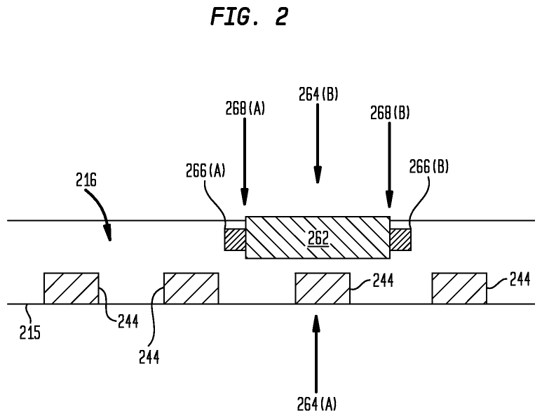

FIG. 2 is a schematic diagram illustrating the techniques presented herein in the context of an example therapeutic substance portion 262 in the form of a solid material (e.g., silicone loaded with a therapeutic substance). More specifically, shown in FIG. 2 is a portion of a stimulating assembly 216 comprising a carrier member 215 with a plurality of stimulating electrodes 244 disposed therein. The illustrated portion of the stimulating assembly 216 also includes the therapeutic substance portion 262 disposed in the carrier member 215. In this example, the stimulating electrodes 244 are located at a first side 264(A) of the carrier member 215, while the therapeutic substance portion 262 is located at a second side 264(B) of the carrier member 215.

Also shown in FIG. 2 are two measurement electrodes, referred to as a first measurement electrode 266(A) and a second measurement electrode 266(B), disposed at first side 268(A) and second side 268(B), respectively, (e.g., at opposing ends) of the therapeutic substance portion 262. The measurement electrodes 266(A) and 266(B) are elements of measurement system (e.g., hardware/circuitry, software, etc.) that is configured to measure the impedance between the measurement electrodes, which indicates the impedance of/across the therapeutic substance portion 262. As described further below, the measurement system is configured to estimate, based on the measured impedance, a state, amount and/or concentration of a therapeutic substance within the therapeutic substance portion 262. As used herein, the term “impedance” refers to electrical impedance (e.g., a measure of the opposition electric current) and can include the electrical impedance of a material to direct current (DC) and/or to alternating current (AC). The impedance can include both resistance and reactance.

As noted, in the example of FIG. 2, the therapeutic substance portion 262 is a solid material that is loaded with a therapeutic substance. An underlying concept of the techniques described below is that the impedance (conductivity/admittance) of a therapeutic substance portion, such as therapeutic substance portion 262, changes as the therapeutic substance moves (e.g., elutes) into the surrounding fluid. In the example of FIG. 2, the therapeutic substances in the therapeutic substance portion 262 are exposed to the ionic fluid (e.g., physiological fluid/saline, which includes water and proteins/ions). As the therapeutic substance starts to leave the therapeutic substance portion 262, it will leave space that can be filled with the ionic fluid, the conductivity of which is higher than the conductivity of the therapeutic substance (e.g., fluid is more conductive than the therapeutic substance). Therefore, as the therapeutic substance leaves the therapeutic substance portion 262, the impedance of the therapeutic substance portion 262, as measured between the first measurement electrode 266(A) and a second measurement electrode 266(B), will decrease. As described elsewhere herein, the impedance of the therapeutic substance portion 262 is monitored via the first measurement electrode 266(A) and a second measurement electrode 266(B).

In accordance with embodiments presented herein, the first measurement electrode 266(A) and a second measurement electrode 266(B) are stable over time to ensure any impedance change is primarily due to a changing drug a state (e.g., amount or concentration) of the therapeutic substance. The stability of the electrodes can be implemented, for example, with a Silicon carbide (SiC) coating on platinum (Pt).

In some embodiments, the interface between the therapeutic substance portion and the electrode(s) 266(A) and/or 266(B) can be prepared to reduce measurement drift and/or other inaccuracies that can be caused by changes in the electrical connection. For example, delamination or changes in contact surface area (e.g., roughening of the therapeutic substance portion from elution) can change the electrical characteristics of the electrode interface.

The therapeutic substance portion can be overmoulded or adhered to the electrode(s) 266(A) and/or 266(B) in a way that promotes a stable electrical interface. The regions of the electrode(s) 266(A) and/or 266(B) that aren't in contact with the therapeutic substance portion can be coated with a material having a higher impedance than the therapeutic substance portion interface to ensure that most of the current is directed through the therapeutic substance portion.

In certain embodiments, the system can be configured to adapt to physical and/or electrical changes that can influence measurement accuracy. For example, the measurement processor can be configured to adapt (e.g., compensate and/or recalibrate) to changes in the electrode interface that are caused by elution of drug from the therapeutic substance portion immediately adjacent to the electrode (e.g., changes in surface roughness and/or contact surface area)

FIG. 2 illustrates an example in which the first measurement electrode 266(A) and a second measurement electrode 266(B) are in physical contact with the therapeutic substance portion 262. It is to be appreciated that, in alternative embodiments, one or both of the first measurement electrode 266(A) and the second measurement electrode 266(B) can be spaced from the therapeutic substance portion 262 (e.g., electrical communication such that at least some, or ideally all, current passes through the therapeutic substance portion). That is, one or both measurement electrodes do not have direct contact to the therapeutic substance (e.g., a stimulating electrode of the electrode array, or one of the two extra-cochlear electrodes). Both electrodes need to be in electrical contact and the therapeutic substance needs to be part of the current path between the two electrodes for the conductivity change to be measurable and, preferably, all (or most) of the current goes through the therapeutic substance portion to obtain the strongest signal (impedance change).

It is also to be appreciated that the specific location of the first measurement electrode 266(A) and the second measurement electrode 266(B) is merely illustrative and that each of the electrodes can have other locations near the therapeutic substance portion 262.

In addition, the use of two electrodes to measure the impedance of the therapeutic substance portion 262 is illustrative of an example two-terminal (two-point) impedance measurement. Other embodiments presented herein can utilize different types of impedance measurements, such as three-terminal (three-point) impedance measurements, four-terminal (four-point) impedance measurements, etc. A three-terminal measurements use a first and second electrode to pass current (current carrying electrodes) and a third, non-current carrying so called “reference electrode,” which is placed in between the first and second electrode. In this arrangement, the impedance change is measured between the first and third electrode. In the four-terminal arrangement, there are first and second current carrying electrodes and third and fourth voltage sensing reference electrodes placed in between the current carrying electrodes. In this arrangement, the impedance is measured between the two reference electrodes. Using reference electrodes can have the benefit of limiting the impact of changes at the electrode to electrolyte interface (electrode surface) to the measured impedance. This can help to ensure impedance changes are dominated by changes in the “bulk” (here the therapeutic substance volume) between the electrodes rather than the complex electrode interfaces. As such, the use of two electrodes for an impedance measurement is merely illustrative and that other embodiments can use additional electrodes.

Moreover, FIG. 2 illustrates a specific arrangement with two dedicated measurement electrodes. The use of two measurement electrodes is merely illustrative and that other arrangements could use, for example, one or more of the stimulating electrodes 244 to perform the impedance measurement. For example, in one such arrangement, current is delivered via measurement electrode 266(A) and returns via one of the stimulating electrodes 244. In this arrangement, measurement electrode 266(B) could be omitted (or used in, for example, a three-terminal or four-terminal measurement).

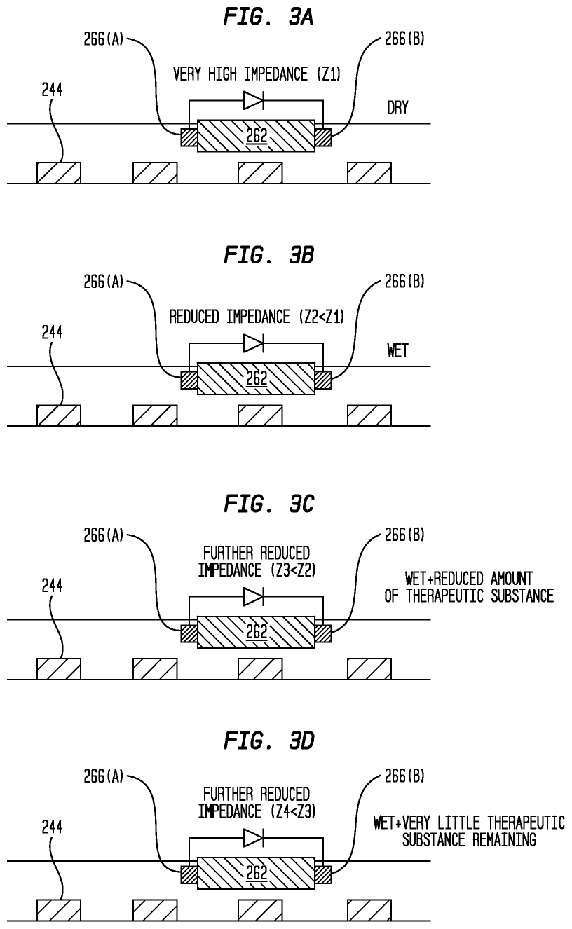

FIGS. 3A-3D are a series of schematic diagrams illustrating the impedance change across the therapeutic substance portion 262. More specifically, FIG. 3A illustrates the impedance ZI of the therapeutic substance portion 262 in a dry state (e.g., pre-implantation), while FIG. 3B illustrates the impedance Z2 of the therapeutic substance portion 262 in an initial wet state (e.g., immediately after implantation). As shown, the impedance of the therapeutic substance portion 262 is high in the dry state (Z1), but the impedance reduces in the initial wet state (Z2<Z1). That is, in a dry state (FIG. 3A), there is a high impedance (Z1) across the therapeutic substance portion 262. There is no reason for current to flow in the dry state (e.g., for silicone with drug molecules, there is no movement of ions). In the initial wet state (FIG. 3B, there is a lower impedance (Z2) across the therapeutic substance portion 262.

FIGS. 3C and 3D illustrate the therapeutic substance portion 262 at two points in time after implantation, namely at point in which some of the therapeutic substance has eluted (FIG. 3C) and at a point in which a small amount of the therapeutic substance remains in the therapeutic substance portion 262. As shown, the impedance Z3, Z4 of the therapeutic substance portion 262 continues to reduce as more and more of the therapeutic substance leaves the therapeutic substance portion 262 and is replaced by the ionic fluid (Z2>Z3>Z4). That is, as the therapeutic substance starts to leave the therapeutic substance portion 262 (FIG. 3C), the therapeutic substance will leave behind space(s), and that space can be filled with ionic fluid (e.g., physiological fluid such as saline), the conductivity of which is higher than the conductivity of the therapeutic substance. Therefore, as the therapeutic substance leaves the therapeutic substance portion 262 (e.g., elutes into perilymph), the impedance across the therapeutic substance should decrease.

In summary, in the examples of FIGS. 2 and 3A-3D, there is a reduced impedance (Z2) from the very high impedance (Z1), but as more therapeutic substance leaves, that impedance will get lower (Z3) and lower (Z4), since the ionic fluid (saline) is more conductive than the therapeutic substance. The more the spaces left behind in the therapeutic substance portion 262 that are filled with the higher-conductivity ionic fluid (saline) as the lower-conductivity therapeutic substance elutes, the lower the impedance will become. Thus, in the case of an uncoated therapeutic substance portion 262, the impedance can decrease over time.

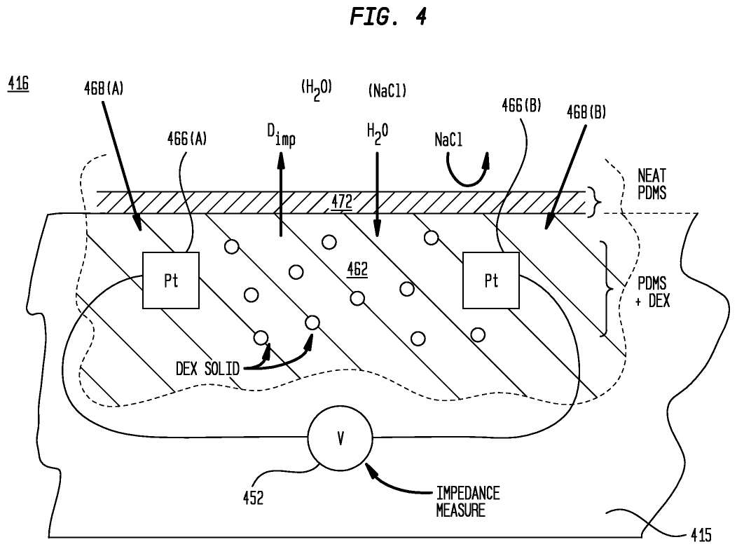

FIG. 4 is a schematic diagram illustrating the techniques presented herein in the context of an example therapeutic substance portion 462 in the form of a solid material (e.g., silicone loaded with a therapeutic substance). More specifically, shown in FIG. 4 is a portion of a stimulating assembly 416 comprising a carrier member 415 with a plurality of stimulating electrodes (not shown in FIG. 4) disposed therein. The illustrated portion of the stimulating assembly 416 also includes the therapeutic substance portion 462 disposed in the carrier member 415.

Also shown in FIG. 4 are two measurement electrodes, referred to as a first measurement electrode 466(A) and a second measurement electrode 466(B), disposed at first side 468(A) and second side 468(B), respectively, (e.g., at opposing ends) of the therapeutic substance portion 462. The measurement electrodes 466(A) and 466(B) are connected to a measurement system (e.g., hardware/circuitry, software, etc.) that is configured to measure the impedance between the measurement electrodes, which indicates the impedance of/across the therapeutic substance portion 462. As described further below, the measurement system is configured to estimate, based on the measured impedance, a state, amount and/or concentration of a therapeutic substance within the therapeutic substance portion 462.

As noted, FIG. 4 shows the use of two measurement electrodes 466(A) and 466(B). In certain embodiments, at least one of the two measurement electrodes could be replaced with a stimulation electrode, such as an intra-cochlear electrode. In one such embodiment, the stimulation electrode is used for both nerve stimulation and as return electrode for measuring the impedance of the therapeutic substance portion 462.

As noted, in the example of FIG. 4, the therapeutic substance portion 462 is a solid material that is loaded with a therapeutic substance. An underlying concept of the techniques described below is that the conductivity (admittance/impedance) of a therapeutic substance portion, such as therapeutic substance portion 462, changes as the therapeutic substance moves (e.g., elutes) into the surrounding fluid. However, in the example shown in FIG. 4, the therapeutic substance portion 462 is covered by a silicone (e.g., PDMS) layer 472. In the case of a silicone-coated therapeutic substance portion 462, the impedance can initially decrease, but subsequent increase over time.

More specifically, in the example of FIG. 4, the spaces/holes/openings created as the therapeutic substance leaves the therapeutic substance portion 462 are filled with pure water (H₂O), since ionic fluid (saline) cannot pass through the silicone layer 472 covering the therapeutic substance portion 462. Pure water is a poorer conductor (as compared to ionic fluid/saline). The silicone layer 472 is an ionic barrier that will not let the salt ions (i.e. sodium (Na⁺) and chlorine (Cl⁻) ions) through, but will let pure water (H₂O) through, while the therapeutic substance will elute out.

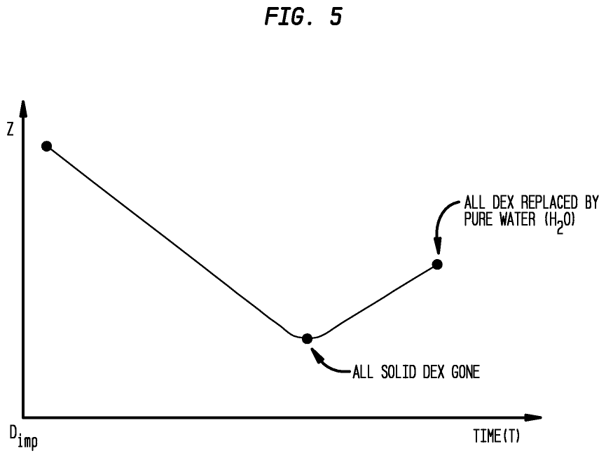

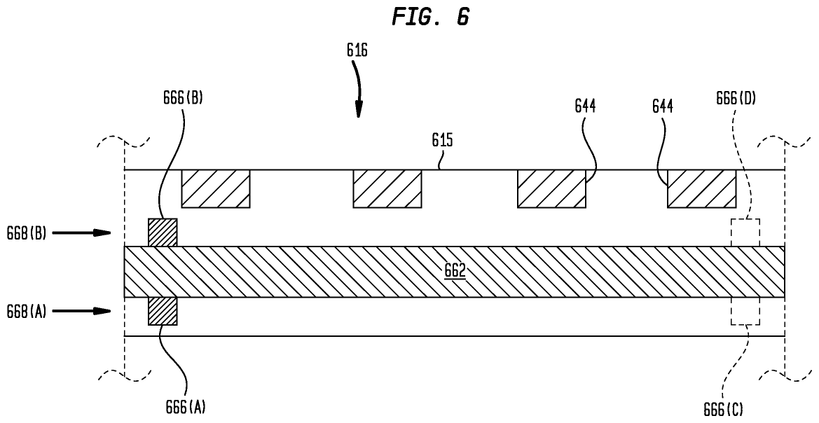

FIG. 5 is a graph generally illustrating further details of the example of FIG. 4, namely a graph illustrating how changing impedance over time can be indicative of the state, amount and/or concentration of therapeutic substance in the therapeutic substance portion. In this example, the water molecules enter the therapeutic substance portion 462 and dissolves the solid therapeutic substance (e.g., Dex) and the solid therapeutic substance is replaced by a saturated therapeutic solution (e.g., water plus the therapeutic substance). Thereafter, the saturated substance is released through the silicone layer 472 and more pure water molecules move in, dissolving more of the solid therapeutic substance. Over time, the volume of solid therapeutic substance reduces, and the volume of spaces filled with saturated therapeutic solution increases. According to this methodology, the impedance decreases over time due to the increasing volume of saturated therapeutic solution in the porous bulk silicone body. The impedance is the lowest when all of the solid therapeutic substance is replaced by the saturated therapeutic solution. The impedance starts to rise again as the remaining therapeutic solution is released. The impedance reaches a second peak once all of the therapeutic solution is gone, and the pores are filled with pure water.

Thus, as described above and further below, some example embodiments of the present disclosure involve measuring the amount of therapeutic substance that has not been eluted so as to thereby determine the amount of the therapeutic substance that has been eluted into the cochlear (e.g., amount of therapeutic substance eluted=total amount of therapeutic substance−amount of therapeutic substance not eluted). This could involve measuring the amount of therapeutic substance that remains in the therapeutic substance portion so as to thereby determine the amount of the therapeutic substance that has been eluted. Some exemplary embodiments involve estimating the state of a therapeutic substance portion from characteristics and/or trends of the measured impedance. For example, a processor can be configured to estimate the status of a therapeutic substance portion from the rate of change of the measured impedance. In some embodiments, the processor can be configured to determine that a therapeutic substance portion is substantially depleted when the measured impedance reaches a steady state (e.g., the rate of change of the measured impedance drops below a threshold value).

FIG. 6 is a schematic diagram illustrating the techniques presented herein in the context of an example therapeutic substance portion 662 in the form of a reservoir (e.g., containing a therapeutic substance in either liquid or solid form). More specifically, shown in FIG. 6 is a portion of a stimulating assembly 616 comprising a carrier member 615 with a plurality of stimulating electrodes 644 disposed therein. The illustrated portion of the stimulating assembly 616 also includes the therapeutic substance portion 662 disposed in the carrier member 615.

Also shown in FIG. 6 are two measurement electrodes, referred to as a first measurement electrode 666(A) and a second measurement electrode 666(B), disposed at first side 668(A) and second side 668(B), respectively, (e.g., at opposing sides) of the therapeutic substance portion 662. The measurement electrodes 666(A) and 666(B) are connected to a measurement system (e.g., hardware/circuitry, software, etc.) that is configured to measure the impedance between the measurement electrodes, which indicates the impedance of/across the therapeutic substance portion 662. As described further below, the measurement system is configured to estimate, based on the measured impedance, a state, amount and/or concentration of a therapeutic substance within the therapeutic substance portion 662. Optionally, as shown with dashed lines in FIG. 6, another pair of measurement electrodes 666(C) and 666(D) can be respectively disposed at the first and second sides of the therapeutic substance portion 662. Alternatively, showing the measurement electrodes 666(C) and 666(D) with dashed lines in FIG. 6 instead represent that there can be different placement locations for the first and second measurement electrodes 666(A) and 666(B) along the therapeutic substance portion 662. Example embodiments are not limited to the number of measurement electrode pairs and/or the location of the measurement electrodes along the sides of the therapeutic substance portion 662.

In the example of FIG. 6, the therapeutic substance portion 662 is a reservoir for a liquid and is a diffusion system. In a diffusion system, the fluid does not physically move, but the therapeutic substance concentration changes over time. That is, there is a concentration gradient movement of the therapeutic substance molecules towards the area of less concentration (which is one end of the reservoir 662 in the illustrated embodiment). Sensors across the reservoir 662 (e.g., measurement electrodes 666A/666B and/or 666C/666D and/or 666A/666C and/or 666B/666D of FIG. 6) can measure impedance at any given point from the reservoir to the delivery site. In certain embodiments, the measurement system could measure impedance at two points across the length to measure a difference. As such, in the example of FIG. 6, the therapeutic substance concentration is changing, which can be measured via a changing impedance, as described elsewhere herein.

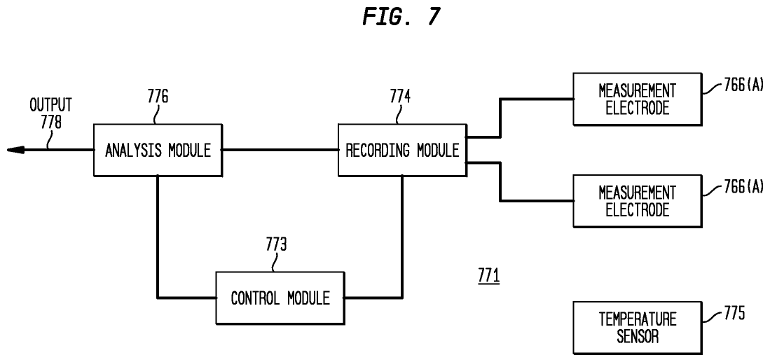

As noted above, aspects of the techniques presented herein capture/record impedance measurements using an impedance measurement system. FIG. 7 is a functional block diagram illustrating an example impedance measurements system 771 configured to implement aspects of the techniques presented herein. The impedance measurements system 771 can be implemented partially or fully, for example, by an implantable medical device (e.g., cochlear implant 102), by the combination of an implantable medical device and an external device (e.g., cochlear implant 102 and external device 110), multiple implantable medical devices, multiple implantable medical devices and multiple external devices, etc.

As shown in FIG. 7, the impedance measurements system 771 comprises a control module 773, at least two measurement electrodes, referred to as measurement electrodes 766(A) and 766(B), a recording module 774, an analysis module 776 and, in certain arrangements, a temperature sensor 775. The analysis module 776, recording module 774, and control module 773 can be implemented in hardware, software, and/or combinations thereof.

In the specific illustrative example of FIG. 7, the recording module 774 is configured to capture/record a plurality of impedances across a therapeutic substance portion. In certain examples, the control module 773 instructs the recording module 774 to perform one or more impedance measurements. The recording module 774 can be configured to, among other operations, amplify the measured impedances, and provide the measured impedances to the analysis module 776.

The analysis module 776 is configured to use the measured impedances to estimate the state, amount, and/or concentration of the therapeutic substance in the subject therapeutic substance portion. In addition, the analysis module 776 can generate one or more outputs 778 representative of the state, amount, and/or concentration. In certain embodiments, the one or more outputs 778 can be audible or visible outputs. In other embodiments, as described further below, the one or more outputs 778 can be control outputs, that are sent to, for example, the control module 773 for use as part of a closed-loop control system.

These example embodiments provide several advantages, including knowing when a therapeutic substance portion is substantially depleted so as to monitor for physiological/electrophysiological changes, knowing when to change out a therapeutic substance portion, when to refill a therapeutic substance portion, and/or to confirm that replacement/replenishment of the therapeutic substance was successful (via a confirmatory change in the conductivity). In general, the therapeutic substance state, amount and/or concentration information could be leveraged in a number of ways to allow a surgeon to optimize or amend a recipient's treatment plan. For example, with a therapeutic substance that has a toxicity limit, the physician can use the therapeutic substance state, amount and/or concentration to determine whether additional systemic administration is possible (if something happens in the ear), while the drug is still being eluted (e.g., if the toxicity limit has been exhausted, the physician might prescribe oral steroids etc.). In relation to refillable therapeutic substance portions, the therapeutic substance state, amount and/or concentration can be used to monitor, for example, rate of delivery, prescribing/information, understanding effects on other electrophysiological measures (e.g., if the 4P impedance started to rise, when the therapeutic substance is there versus when there is no therapeutic substance it might mean different things), etc.

In some embodiments, the measured impedance/conductivity can be dependent upon temperature (i.e., impedance can vary widely as temperature changes). Therefore, a temperature sensor 775 (FIG. 7) can also be provided near a therapeutic substance portion, and the temperature recorded by the temperature sensor 775 can be used to compensate the impedance measurement to account for varying temperature. The temperature sensor can be a resistive wire, for example. In embodiments where the temperature of tissue around the therapeutic substance portion is expected to vary (e.g. subcutaneous devices), the measured impedance can be inaccurate without controlling for temperature. Therefore, the temperature measurement can be used to calibrate/compensate/correct the impedance measurement across the therapeutic substance portion (e.g., analyze the conductivity). The temperature sensor could also be used for other purposes, such as temperature monitoring, local inflammation temp monitoring, etc.

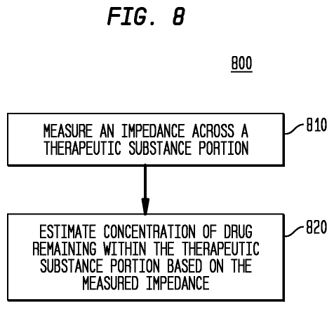

In connection with the various example embodiments described above with reference to various methods will now be described below with reference to FIGS. 8, 9, 10, and 11.

FIG. 8 shows an example method 800 for monitoring therapeutic substance state, amount and/or concentration in a therapeutic substance portion using a measurement system according to an aspect of the present disclosure. In particular, a measurement system measures an impedance across a therapeutic substance portion (Step 810) and a state, amount and/or concentration of the therapeutic substance remaining within the therapeutic substance portion is estimated based on the measured impedance (Step 820).

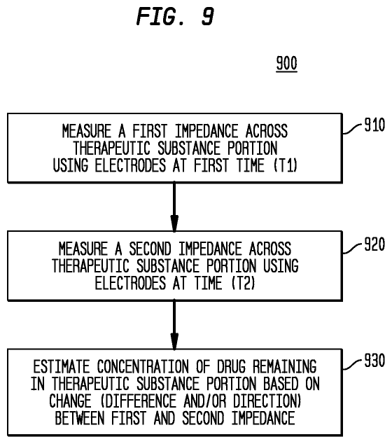

FIG. 9 shows an example method 900 for monitoring therapeutic substance state, amount and/or concentration in a therapeutic substance portion using a measurement system according to another aspect of the present disclosure. In this variation, the measurement electrodes associated with a therapeutic substance portion are used to measure a first impedance across the therapeutic substance portion at a first time (T1) (Step 910). The measurement electrodes associated with the therapeutic substance portion are then used to measure a second impedance across the therapeutic substance portion at a second time (T2) (Step 920). The state, amount and/or concentration of therapeutic substance remaining within the therapeutic substance portion is estimated based on a change between the first measured impedance at time Tl and the second measured impedance at time T2 (Step 930). To estimate the state, amount and/or concentration of therapeutic substance remaining in this variation, the first and second impedance values can be compared, and a difference between the pair can be determined (along with a direction of the change, increasing vs. decreasing). The rate of change and/or the amount of change can be indicative of therapeutic substance concentration left in the therapeutic substance portion after a certain period of time.

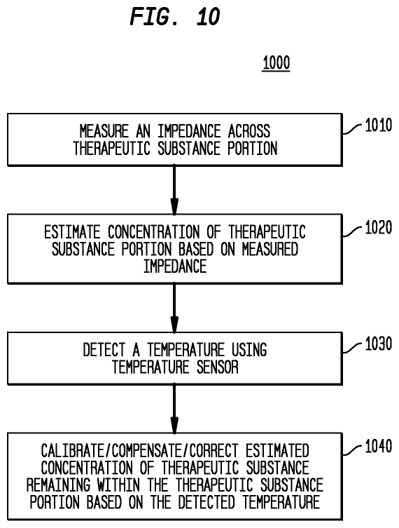

FIG. 10 shows an example method 1000 for monitoring therapeutic substance state, amount and/or concentration in a therapeutic substance portion using an impedance measurement system (e.g., hardware/circuitry, software, etc.) and a temperature sensor according to an aspect of the present disclosure. In particular, the measurement system measures an impedance across a therapeutic substance portion (Step 1010) and a state, amount and/or concentration of the therapeutic substance remaining within the therapeutic substance portion is estimated based on the measured impedance (Step 1020).

In this variation, method 1000 further includes detecting a temperature within the proximity to the therapeutic substance portion using the temperature sensor (Step 1030), and calibrating/compensating/correcting the estimated state, amount and/or concentration of therapeutic substance remaining within the therapeutic substance portion based on the detected temperature (Step 1040).

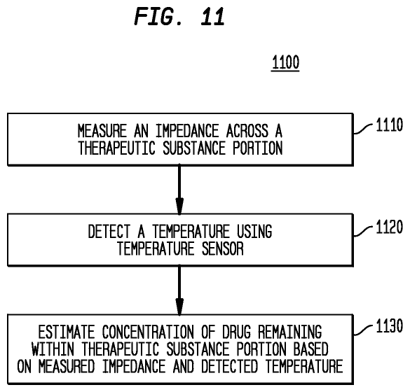

FIG. 11 shows another example method 1100 for monitoring therapeutic substance state, amount and/or concentration in a therapeutic substance portion using an impedance measurement system (e.g., hardware/circuitry, software, etc.) and a temperature sensor according to another aspect of the present disclosure. In particular, the measurement system measures an impedance across a therapeutic substance portion (Step 1110) and, in this variation, detects a temperature within the proximity to the therapeutic substance portion using the temperature sensor (Step 1120). The measurement system then estimates the state, amount and/or concentration of the therapeutic substance remaining within the therapeutic substance portion based on the measured impedance and the detected temperature (Step 1130).

In methods 1000 and 1100 above, various adjustments can be made to increase or decrease the measured impedance to account for temperature in order to make the therapeutic substance state, amount and/or concentration estimate more accurate (because impedance can often vary at higher and lower temperatures).

Some other example embodiments will now be described with further explanation details. One example embodiment just uses a water-soluble molecule coating (could be a therapeutic substance or just an indicator) on an electrode, either within a reservoir or on an electrode on the outer surface of the electrode array. An outer surface electrode is used to determine the state, amount and/or concentration of therapeutic substance in the cochlear, which in turn can be used to infer the amount of therapeutic substance delivered. The molecule leaves from a layer placed on the electrode when the concentration in the cochlea (or reservoir) reaches some target level. For example, an electrode close to the therapeutic substance delivery point could be used, as this will ensure a high concentration near the layer (initially). The measurement could be made, for example, between an intra-cochlear electrode and an extra-cochlear electrode. The therapeutic substance or other water-soluble molecule could be alone or loaded into a polymer matrix. In addition, a matrix can include different water-soluble molecules with different exit. For example, a molecule that is not released until the therapeutic substance in the reservoir is at 10% (or some other threshold value) could be used.

Another example embodiment uses a therapeutic substance containing coating (e.g., dex eluting silicone coating) on one or two electrodes inside a reservoir filled with therapeutic substance solution. The therapeutic substance in the coating stays solid so long as the concentration of the solution is at the solubility limit at a given temperature (e.g., 37 degrees). In this state, the impedance between the two electrodes is stable over time. As the therapeutic substance is released from the reservoir, the concentration of the solution decreases below the solubility limit and some amount of solid therapeutic substance from the coating will go into the solution to keep the solution to compensate (e.g., keep the solution near the solubility limit concentration). This will decrease the impedance between the two electrodes. In this scenario, the impedance change is a measure of how much therapeutic substance has been released from the reservoir into the body. The solid therapeutic substance acts as a reservoir itself. When all solid therapeutic substance in the coating is dissolved, no significant impedance change will be measured, but the solution in the reservoir is still at the same concentration (solubility limit). In that final phase, therapeutic substance release into the body will go on without seeing a significant impedance change anymore until all therapeutic substance has left the device. This extends the concept to (indirect) measures of liquid therapeutic substance concentration.

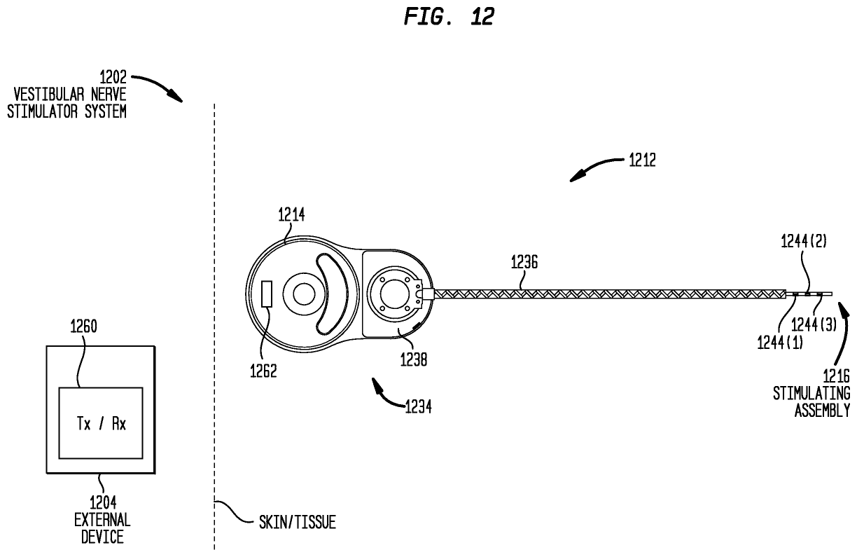

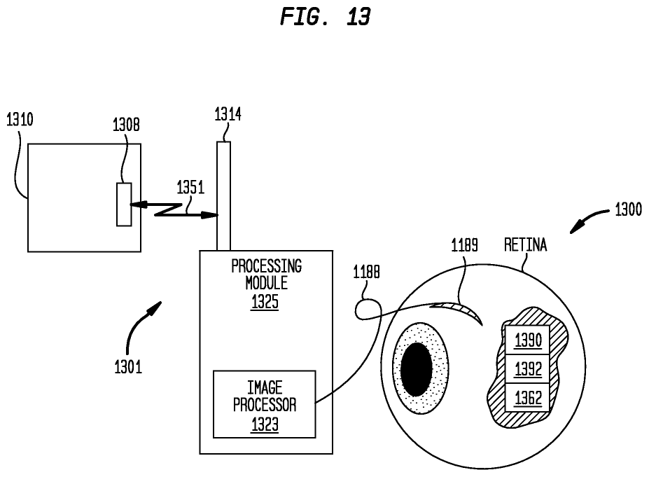

As previously described, the technology disclosed herein can be applied in any of a variety of circumstances and with a variety of different devices. Example devices that can benefit from technology disclosed herein are described in more detail in FIGS. 12 and 13, below.

FIG. 12 illustrates an example vestibular stimulator system 1202, with which embodiments presented herein can be implemented. As shown, the vestibular stimulator system 1202 comprises an implantable component (vestibular stimulator) 1212 and an external device/component 1204 (e.g., external processing device, battery charger, remote control, etc.). The external device 1204 comprises a transceiver unit 1260. As such, the external device 1204 is configured to transfer data (and potentially power) to the vestibular stimulator 1212.

The vestibular stimulator 1212 comprises an implant body (main module) 1234, a lead region 1236, and a stimulating assembly 1216, all configured to be implanted under the skin/tissue (tissue) of the recipient. The implant body 1234 generally comprises a hermetically-sealed housing 1238 in which RF interface circuitry, one or more rechargeable batteries, one or more processors, and a stimulator unit are disposed. The implant body 1234 also includes an internal/implantable coil 1214 that is generally external to the housing 1238, but which is connected to the transceiver via a hermetic feedthrough (not shown).

The stimulating assembly 1216 comprises a plurality of electrodes 1244(1)-(3) disposed in a carrier member (e.g., a flexible silicone body). In this specific example, the stimulating assembly 1216 comprises three (3) stimulation electrodes, referred to as stimulation electrodes 1244(1), 1244(2), and 1244(3). The stimulation electrodes 1244(1), 1244(2), and 1244(3) function as an electrical interface for delivery of electrical stimulation signals to the recipient's vestibular system.

The stimulating assembly 1216 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

As shown, the implant body 1234 also comprises one or more therapeutic substance delivery portions 1262. The therapeutic substance delivery portion(s) 1262 include/comprise one or more therapeutic substances for delivery to the recipient. The implant body 1234 also includes a measurement system (not shown in FIG. 12), as described elsewhere herein, configured to measure impedance(s) across the one or more therapeutic substance delivery portions 1262 and to use the impedances to determine the state, amount and/or concentration of the therapeutic substance(s) in the one or more therapeutic substance delivery portions 1262.

FIG. 13 illustrates a retinal prosthesis system 1301 that comprises an external device 1310 configured to communicate with a retinal prosthesis 1300 via signals 1351. The retinal prosthesis 1300 comprises an implanted processing module 1325 and a retinal prosthesis sensor-stimulator 1390 is positioned proximate the retina of a recipient. The external device 1310 and the processing module 1325 can communicate via coils 1308 and 1314.

In an example, sensory inputs (e.g., photons entering the eye) are absorbed by a microelectronic array of the sensor-stimulator 1390 that is hybridized to a glass piece 1392 including, for example, an embedded array of microwires. The glass can have a curved surface that conforms to the inner radius of the retina. The sensor-stimulator 1390 can include a microelectronic imaging device that can be made of thin silicon containing integrated circuitry that convert the incident photons to an electronic charge.

The processing module 1325 includes an image processor 1323 that is in signal communication with the sensor-stimulator 1390 via, for example, a lead 1388 which extends through surgical incision 1389 formed in the eye wall. In other examples, processing module 1325 is in wireless communication with the sensor-stimulator 1390. The image processor 1323 processes the input into the sensor-stimulator 1390, and provides control signals back to the sensor-stimulator 1390 so the device can provide an output to the optic nerve. That said, in an alternate example, the processing is executed by a component proximate to, or integrated with, the sensor-stimulator 1390. The electric charge resulting from the conversion of the incident photons is converted to a proportional amount of electronic current which is input to a nearby retinal cell layer. The cells fire and a signal is sent to the optic nerve, thus inducing a sight perception.

The processing module 1325 can be implanted in the recipient and function by communicating with the external device 1310, such as a behind-the-ear unit, a pair of eyeglasses, etc. The external device 1310 can include an external light/image capture device (e.g., located in/on a behind-the-ear device or a pair of glasses, etc.), while, as noted above, in some examples, the sensor-stimulator 1390 captures light/images, which sensor-stimulator is implanted in the recipient.

As shown, the one or more therapeutic substance delivery portions 1362 can be implanted in the recipient. The therapeutic substance delivery portion(s) 1362 include/comprise one or more therapeutic substances for delivery to the recipient. The retinal prosthesis system 1301 also includes a measurement system (not shown in FIG. 13), as described elsewhere herein, configured to measure impedance(s) across the one or more therapeutic substance delivery portions 1362 and to use the impedances to determine the state, amount and/or concentration of the therapeutic substance(s) in the one or more therapeutic substance delivery portions 1362.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A system, comprising:
at least one therapeutic substance portion including a therapeutic substance; and
a measurement system configured to measure an impedance across the at least one therapeutic substance portion and to estimate a state of the therapeutic substance within the at least one therapeutic substance portion based on the measured impedance across the at least one therapeutic substance portion.

2. The system of claim 1, wherein the measurement system includes at least a first electrode and a second electrode disposed at opposing sides of the at least one therapeutic substance portion.

3. The system of claim 2, wherein the first electrode is in physical contact with the at least one therapeutic substance portion, and the second electrode is physically separated from the at least one therapeutic substance portion.

4. The system of claim 2, wherein the second electrode is an electrode that is configured to deliver electrical stimulation signals to tissue of a recipient.

5. The system of claim 2, wherein the first electrode and the second electrode are each in physical contact with the at least one therapeutic substance portion.

6. The system of claim 2, wherein the at least one therapeutic substance portion comprises silicone loaded with the therapeutic substance.

7. The system of claim 2, wherein the first electrode and the second electrode are configured to be stable over time.

8. The system of claim 1, wherein the measurement system includes a first pair of opposing electrodes disposed across a first section of the at least one therapeutic substance portion and a second pair of opposing electrodes disposed across a second section of the at least one therapeutic substance portion.

9. The system of claim 1, further comprising a temperature sensor configured to detect a temperature within a proximity to the at least one therapeutic substance portion.

10. The system of claim 1, wherein the measurement system is configured to estimate a concentration of the therapeutic substance within the at least one therapeutic substance portion.

11. The system of claim 9, wherein the measurement system is configured to estimate the concentration of the therapeutic substance within the at least one therapeutic substance portion based on the measure an impedance across the at least one therapeutic substance portion and the temperature detected by the temperature sensor within the proximity to the at least one therapeutic substance portion.

12. The system of claim 1, wherein the at least one therapeutic substance portion is disposed in, and substantially surrounded by, a polymer body, and includes at least one exposed surface.

13. The system of claim 1, wherein the at least one therapeutic substance portion is disposed in, and substantially surrounded by, a polymer body, and includes a first surface, and wherein a polymer layer is disposed on the first surface.

14. The system of claim 13, wherein polymer layer includes one or more openings.

15. The system of claim 1, wherein the at least one therapeutic substance portion is a solid polymer loaded with at least one therapeutic substance.

16. The system of claim 1, wherein the at least one therapeutic substance portion is a reservoir containing at least one fluid therapeutic substance.

17-31. (canceled)

32. One or more non-transitory computer readable storage media comprising instructions that, when executed by a processor, cause the processor to:
obtain an impedance of at least one therapeutic substance portion; and
estimate, based on the impedance of the at least one therapeutic substance portion, an amount of at least one therapeutic substance remaining within the at least one therapeutic substance portion.

33. The one or more non-transitory computer readable storage media of claim 32, wherein the instructions that, when executed, cause the processor to obtain an impedance of at least one therapeutic substance portion include instructions that cause the processor to:
measure the impedance of at least one therapeutic substance portion using at least a first electrode and at least a second electrode positioned proximate the at least one therapeutic substance portion.

34. The one or more non-transitory computer readable storage media of claim 32, wherein the instructions that, when executed, cause the processor to obtain an impedance of at least one therapeutic substance portion include instructions that cause the processor to:
measure the impedance of at least one therapeutic substance portion using at least a first electrode, at least a second electrode, and at least a third electrode positioned proximate the at least one therapeutic substance portion.

35. The one or more non-transitory computer readable storage media of claim 32, wherein the instructions that, when executed, cause the processor to obtain an impedance of at least one therapeutic substance portion include instructions that cause the processor to:
measure the impedance of at least one therapeutic substance portion using at least a first electrode, at least a second electrode, at least a third electrode, and at least a fourth electrode positioned proximate the at least one therapeutic substance portion.

36. The one or more non-transitory computer readable storage media of claim 32, further comprising instructions executable to:
obtain a temperature measured within a proximity to the at least one therapeutic substance portion; and
estimate the amount of at least one therapeutic substance remaining within the at least one therapeutic substance portion further based on the temperature.

37. (canceled)
